(19) United States
(12) Patent Application Publication
(10) Pub. No.: US 2018/0359955 A1
(43) Pub. Date: Dec. 20, 2018

(54) SYSTEMS AND METHODS FOR SELF-LEARNING IN A GROW POD

(71) Applicant: Grow Solutions Tech LLC, Lehi, UT (US)

(72) Inventor: Gary Bret Millar, Highland, UT (US)

(21) Appl. No.: 15/970,582

(22) Filed: May 3, 2018

Related U.S. Application Data
(60) Provisional application No. 62/519,318, filed on Jun. 14, 2017.
(60) Provisional application No. 62/519,304, filed on Jun. 14, 2017.

(51) Int. Cl. A01G 9/26; H04W 4/38; G06N 3/08; H04L 29/08; G01N 33/00

(52) U.S. Cl. CPC A01G 9/26; H04W 4/38; G01N 33/0098; H04L 67/125; G06N 3/08

(57) ABSTRACT

Embodiments described herein include systems and methods for self-learning in a grow pod. One embodiment includes a cart that houses a plant for growth, a track that receives the cart, where the track causes the cart to traverse the assembly line grow pod along a predetermined path, and an environmental affecter for providing sustenance to the plant. Some embodiments include a sensor for monitoring an output of the plant and a computing device. The computing device may store logic that causes the assembly line grow pod to receive growth data from the sensor to determine the output of the plant and compare the output of the plant against an expected plant output. In some embodiments, the logic causes the assembly line grow pod to determine an alteration to a grow recipe to improve the output of the plant and alter the grow recipe for improving the output of the plant.

### CROSS REFERENCE

This application claims the benefit of U.S. Provisional Application Ser. No. 62/519,318 and U.S. Provisional Application Ser. No. 62/519,304, both of which are incorporated by reference in their entireties.

### TECHNICAL FIELD

Embodiments described herein generally relate to systems and methods for self-learning in an industrial grow pod and, more specifically, to embodiments that are configured to utilize a grow recipe for a grow pod and alter the grow recipe, based on analysis of plant growth.

### BACKGROUND

While crop growth technologies have advanced over the years, there are still many problems in the farming and crop industry today. As an example, while technological advances have increased efficiency and production of various crops, many factors may affect a harvest, such as weather, disease, infestation, and the like. Additionally, while the United States currently has suitable farmland to adequately provide food for the U.S. population, other countries and future populations may not have enough farmland to provide the appropriate amount of food.

Additionally, while greenhouses typically provide shelter of plants from the elements and potentially have watering systems, these current solutions are typically unable to change, based on achieved results. As such, these current solutions typically do not provide any mechanism for improving.

### SUMMARY

Embodiments described herein include systems and methods for self-learning in a grow pod. One embodiment includes a cart that houses a plant for growth, a track that receives the cart, where the track causes the cart to traverse the assembly line grow pod along a predetermined path, and an environmental affecter for providing sustenance to the plant. Some embodiments include a sensor for monitoring an output of the plant and a computing device. The computing device may store logic that causes the assembly line grow pod to receive growth data from the sensor to determine the output of the plant and compare the output of the plant against an expected plant output. In some embodiments, the logic causes the assembly line grow pod to determine an alteration to a grow recipe to improve the output of the plant and alter the grow recipe for improving the output of the plant.

Some embodiments of a system for self-learning in a grow pod include a tray that receives a plurality of seeds and for growing the plurality of seeds into respective plants, an environmental affecter for providing sustenance to the plurality of seeds, and a sensor for monitoring a plant output. Some embodiments include a computing device that stores logic that causes the system to receive growth data from the sensor to determine the plant output and compare the plant output against expected plant output. In some embodiments, the logic causes the system to determine an alteration to a grow recipe to improve the plant output and alter the grow recipe for improving the plant output and for improving a plant output of future plants.

Additionally, some embodiments of a system include an assembly line grow pod that includes a cart that houses a plant for growth, a track that receives the cart, where the track causes the cart to traverse the assembly line grow pod along a predetermined path, and an environmental affecter for providing sustenance to the plant. Some embodiments include a sensor for monitoring an output of the plant and a computing device that stores logic. The logic may cause the system to receive growth data from the sensor to determine the output of the plant, compare the output of the plant against expected plant output, and determine an alteration to a grow recipe to improve the output of a future plant. In some embodiments, the logic causes the system to alter the grow recipe for improving the output of the output of the future plant.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 depicts an assembly line grow pod for self-learning, according to embodiments described herein;

FIG. 2 depicts a computing environment for a self-learning in a grow pod, according to embodiments described herein;

FIG. 3 depicts a computing device for self-learning in a grow pod, according to embodiments described herein;

FIG. 4 depicts a neural network node configuration for self-learning in a grow pod, according to embodiments described herein;

FIG. 5 depicts a flowchart for self-learning in a grow pod, according to embodiments described herein; and

FIG. 6 depicts a flowchart for self-learning and adjusting a grow recipe, according to embodiments described herein.

### DETAILED DESCRIPTION

Embodiments disclosed herein include systems and methods for self-learning in a grow pod. Some embodiments of a grow pod may include a computing device that determines or receives a grow recipe. The grow recipe may be configured to actuate one or more environmental affecters, such as components associated with watering, lighting, nutrient, temperature, pressure, molecular air content, humidity, airflow, etc. As an example, environmental affecters may include a light source, a watering device, a nutrient dispensing device, a temperature control device, a humidity control device, a pressure control device, an airflow control device, and/or other device for adjusting the environment of the grow pod and/or affecting output of a plant.

If a microgreen is being grown, the grow recipe may indicate that a blue wavelength of light is applied to the plant for a predetermined time or growth. The recipe may also provide a set watering schedule and/or a watering schedule based on water absorption of the plant. Depending on the embodiment, the grow recipe may be designed such that the system is adaptive to changes in the plant output. If the plant does not absorb all of the provided water, the grow recipe may reduce the amount of water applied to the plant. Similarly, the recipe may not provide an exact time for harvesting, but may instead cause harvesting based on a developmental stage of the plant being reached. Accordingly, the recipe may be utilized for growing and harvesting the plant.

However, some embodiments of the grow recipe may not be capable of fully adapting to all situations as written. As such, embodiments described herein may be configured with one or more sensors to determine plant output, such plant growth, root growth, leaf growth, stalk growth, fruit growth, flower growth, protein production, chlorophyll production, seed success rate and/or other factors of the plant to determine how the plant has grown under the grow recipe. If the plant is deficient in an output measurement (such as height, girth, fruit output, water consumption, light consumption, etc.), the embodiments described herein may utilize a neural network to change the recipe to correct that deficiency. Similarly, if the plant exceeds expectation for a particular measurement, the neural network may be utilized to determine the cause of the unexpected result and make changes to the recipe to reproduce the unexpected result. The systems and methods for self-learning in a grow pod incorporating the same will be described in more detail, below.

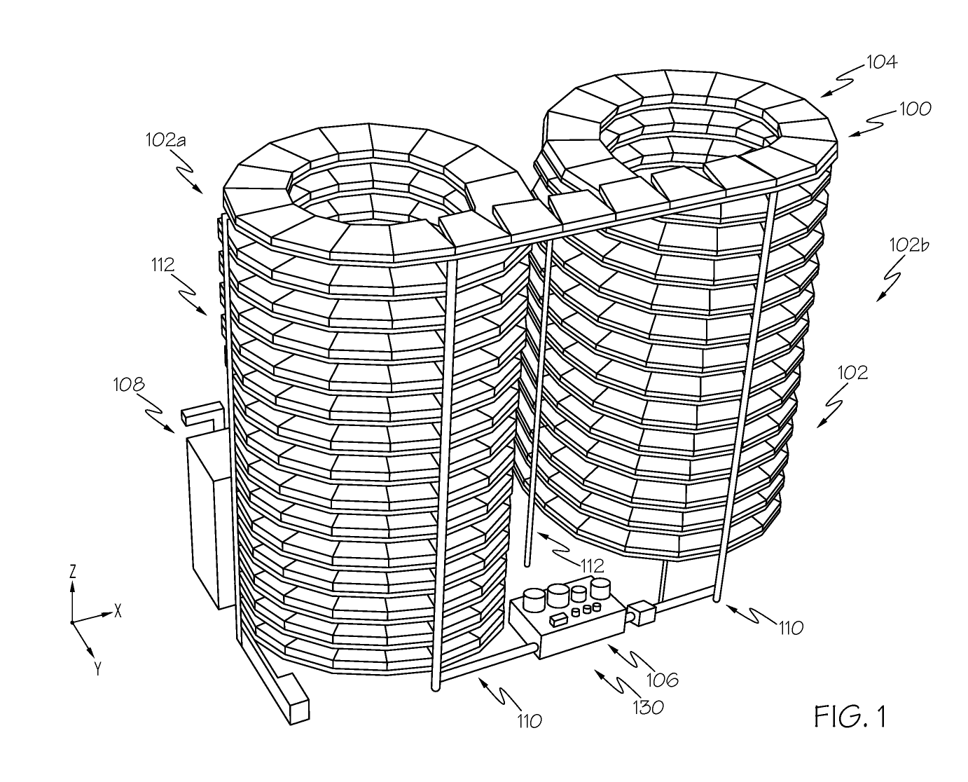

Referring now to the drawings, FIG. 1 depicts a grow pod 100 for self-learning, according to embodiments described herein. As illustrated, the grow pod 100 may be configured as an assembly line grow pod and thus may include a track 102 that holds one or more carts 104. The track 102 may include an ascending portion 102a, a descending portion 102b, and a connection portion 102c. The track 102 may wrap around (in a counterclockwise direction in FIG. 1) a first axis such that the carts 104 ascend upward in a vertical direction. The connection portion 102c may be relatively level (although this is not a requirement) and is utilized to transfer carts 104 to the descending portion 102b. The descending portion 102b may be wrapped around a second axis (again in a counterclockwise direction in FIG. 1) that is substantially parallel to the first axis, such that the carts 104 may be returned closer to ground level. Another connection portion may also be included to complete the circuit of the track 102 and allow carts 104 on the track 102 to begin another cycle.

The grow pod 100 may also include one or more environment affecters. As an example, the grow pod 100 may also include a plurality of lighting devices, such as light emitting diodes (LEDs). The lighting devices may be disposed on and/or adjacent the track 102, such that the lighting devices direct photons to the plants residing on the carts 104. In some embodiments, the lighting devices are configured to create a plurality of different colors and/or wavelengths of light, depending on the application, the type of plant being grown, and/or other factors. While in some embodiments, LEDs are utilized for this purpose, this is not a requirement. Any lighting device that produces low heat and provides the desired functionality may be utilized.

Also depicted in FIG. 1 is a master controller 106 and other environment affecters, such as a seeder component 108, a nutrient dosing component, a water distribution component, an air distribution component, and/or other hardware for controlling various components of the grow pod 100. The master controller 106 may include a computing device 130, which is described in more detail below.

The seeder component 108 may be configured to seed one or more carts 104 as the carts 104 pass the seeder in the assembly line. Depending on the particular embodiment, each cart 104 may include a tray, such as a single section tray for receiving a plurality of seeds. Some embodiments may include a multiple section tray for receiving individual seeds (or a plurality of seeds) in each section (or cell). In the embodiments with a single section tray, the seeder component 108 may detect presence of the respective cart 104 and may begin laying seed across an area of the single section tray. The seed may be laid out according to a desired depth of seed, a desired number of seeds, a desired surface area of seeds, and/or according to other criteria. In some embodiments, the seeds may be pre-treated with nutrients and/or anti-buoyancy agents (such as water) as these embodiments may not utilize soil to grow the seeds and thus might need to be submerged.

In the embodiments where a multiple section tray is utilized with one or more of the carts 104, the seeder component 108 may be configured to individually insert one or more seeds into one or more of the sections of the tray. Again, the seeds may be distributed on the tray (or into individual cells) according to a desired number of seeds, a desired area the seeds should cover, a desired depth of seeds, etc.

The watering component may be coupled to one or more water lines 110, which distribute water and/or nutrients to one or more trays at predetermined areas of the grow pod 100. In some embodiments, seeds may be sprayed with water or other liquid to reduce buoyancy and then may be flooded. Additionally, water usage and consumption may be monitored, such that at subsequent watering stations, this data may be utilized to determine an amount of water to apply to a seed at that time.

Also depicted in FIG. 1 are airflow lines 112. Specifically, the master controller 106 may include and/or be coupled to one or more components (such as air ducts) that delivers airflow for temperature control, pressure, carbon dioxide control, oxygen control, nitrogen control, etc. Accordingly, the airflow lines 112 may distribute the airflow at predetermined areas in the grow pod 100.

Additionally, the grow pod 100 may include one or more output sensors for monitoring light that a plant receives, light absorbed by a plant, water received by a plant, water absorbed by a plant, nutrients received by a plant, water absorbed by a plant, environmental conditions provided to a plant, and/or other system outputs. Depending on the particular type of output data being monitored, the sensors may include cameras, light sensors, weight sensors, color sensors, proximity sensors, sound sensors, moisture sensors, heat sensors, etc. Similarly, growth sensors may be included in the grow pod 100, which may be configured to determine height of a plant, width (or girth) of a plant, fruit output of a plant, root growth of a plant, weight of a plant, etc. As such, the growth sensors may include cameras, weight sensors, proximity sensors, color sensors, light sensors, etc.

It should be understood that while the embodiment of FIG. 1 depicts an assembly line grow pod that wraps around a plurality of axes, this is merely one example. Any configuration of assembly line or stationary grow pod may be utilized for performing the functionality described herein. Additionally, while two helical structures are depicted, more ore fewer may be utilized, depending on the embodiment.

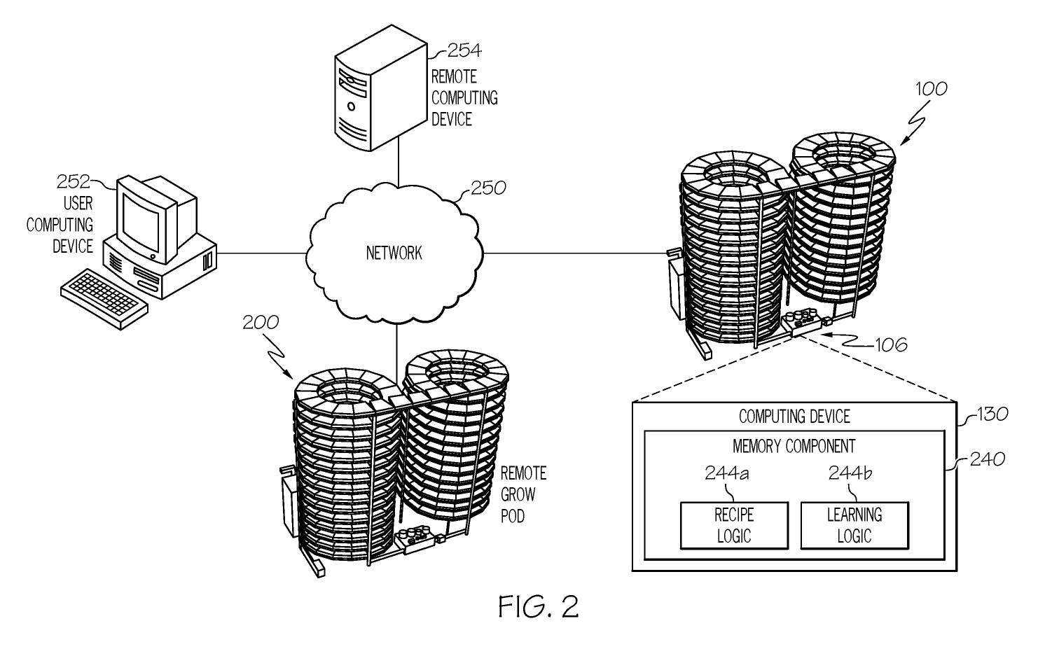

FIG. 2 depicts a computing environment for a self-learning in a grow pod 100, according to embodiments described herein. As illustrated, the grow pod 100 may include a master controller 106, which may include a computing device 130. The computing device 130 may include a memory component 240, which stores recipe logic 244a and learning logic 244b. As described in more detail below, the recipe logic 244a may receive and/or determine one or more grow recipes for growing a plant. Specifically, the recipe logic 244a may be configured to cause the computing device 130 to actuate watering, light, nutrient, environment, and/or other system components for providing nourishment to the plant. The recipe logic 244a may also receive data from the output sensors and the growth sensors for determining growth of the plants that utilize the recipe.

Similarly, the learning logic 244b may be configured as a neural network or other logic to determine an expectation of one or more aspects of plant growth and compare those expectations to the actual plant growth. If the actual plant growth exceeds the expectation, the learning logic 244b may cause the computing device 130 to alter the recipe logic 244a to achieve the unexpected result. Similarly, if the actual plant growth did not exceed the expectation, the learning logic 244b may cause the computing device 130 to determine a modification to the recipe logic 244a to improve the actual plant growth for future plants and implement that change.

Additionally, the grow pod 100 is coupled to a network 250. The network 250 may include the internet or other wide area network, a local network, such as a local area network, a near field network, such as Bluetooth or a near field communication (NFC) network. The network 250 is also coupled to a remote grow pod 200, a user computing device 252, and/or a remote computing device 254. The remote grow pod 200 may be configured similar to the grow pod 100, but need not be a duplicate. Regardless, the remote grow pod 200 may run the same or similar recipes as the grow pod 100 and thus may learn adjustments to the recipe for improved results. Accordingly, the remote grow pod 200 may communicate with the grow pod 100 (and vice versa) to share learned knowledge and/or revised recipes.

The user computing device 252 may include a personal computer, laptop, mobile device, tablet, server, etc. and may be utilized as an interface with a user. As an example, a user may send a recipe or alteration to a recipe to the computing device 130 for implementation by the grow pod 100. Another example may include the grow pod 100 sending notifications to a user of the user computing device 252.

Similarly, the remote computing device 254 may include a server, personal computer, tablet, mobile device, etc. and may be utilized for machine to machine communications. As an example, if the grow pod 100 determines a type of seed being used (and/or other information, such as ambient conditions), the computing device 130 may communicate with the remote computing device 254 to retrieve a previously stored recipe or alteration of a recipe for those conditions. As such, some embodiments may utilize an application program interface (API) to facilitate this or other computer-to-computer communications. Similarly, while some embodiments may be configured such that the computing device 130 learns successful changes to a recipe, this is just an example. Some embodiments may be configured such that the learning logic 244b (or other learning logic) is executed by the remote computing device 254 and then communicated to the grow pod 100 and/or remote grow pod 200 for implementation.

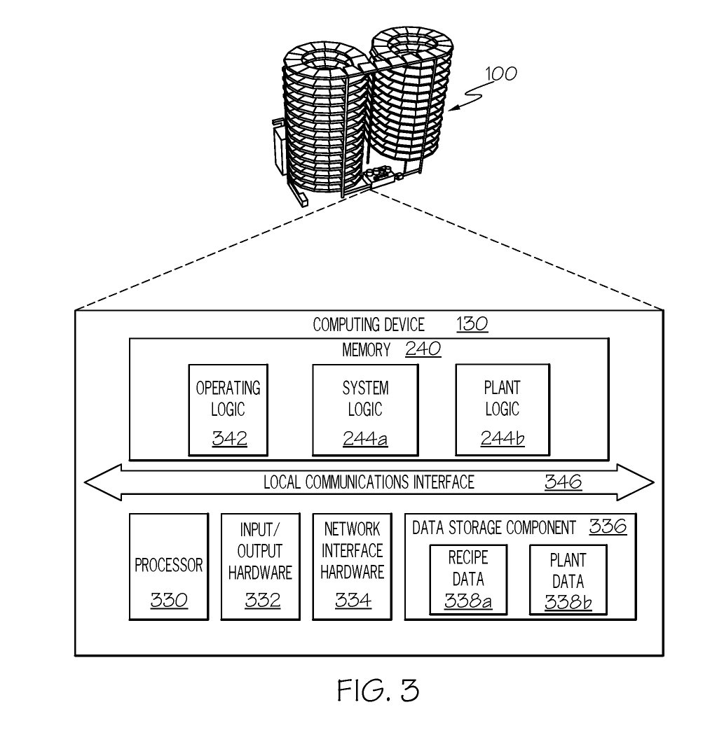

FIG. 3 depicts a computing device 130 for self-learning in a grow pod 100, according to embodiments described herein. As illustrated, the computing device 130 includes a processor 330, input/output hardware 332, the network interface hardware 334, a data storage component 336 (which stores recipe data 338a, plant data 338b, and/or other data), and the memory component 240. The memory component 240 may be configured as volatile and/or nonvolatile memory and as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the computing device 130 and/or external to the computing device 130.

The memory component 240 may store operating logic 342, the recipe logic 244a, and the learning logic 244b. The recipe logic 244a and the learning logic 244b may each include a plurality of different pieces of logic, each of which may be embodied as a computer program, firmware, and/or hardware, as an example. A local interface 346 is also included in FIG. 3 and may be implemented as a bus or other communication interface to facilitate communication among the components of the computing device 130.

The processor 330 may include any processing component operable to receive and execute instructions (such as from a data storage component 336 and/or the memory component 140). The input/output hardware 332 may include and/or be configured to interface with microphones, speakers, a display, and/or other hardware.

The network interface hardware 334 may include and/or be configured for communicating with any wired or wireless networking hardware, including an antenna, a modem, LAN port, wireless fidelity (Wi-Fi) card, WiMax card, ZigBee card, Bluetooth chip, USB card, mobile communications hardware, and/or other hardware for communicating with other networks and/or devices. From this connection, communication may be facilitated between the computing device 130 and other computing devices, such as a computing device 130 on the remote grow pod 200, the user computing device 252, and/or remote computing device 254.

The operating logic 342 may include an operating system and/or other software for managing components of the computing device 130. As also discussed above, the recipe logic 244a and the learning logic 244b may reside in the memory component 240 and may be configured to perform the functionality, as described herein.

It should be understood that while the components in FIG. 3 are illustrated as residing within the computing device 130, this is merely an example. In some embodiments, one or more of the components may reside external to the computing device 130. It should also be understood that, while the computing device 130 is illustrated as a single device, this is also merely an example. In some embodiments, the recipe logic 244a and the learning logic 244b may reside on different computing devices. As an example, one or more of the functionalities and/or components described herein may be provided by the remote grow pod 200, the user computing device 252, and/or remote computing device 254.

Additionally, while the computing device 130 is illustrated with the recipe logic 244a and the learning logic 244b as separate logical components, this is also an example. In some embodiments, a single piece of logic (and/or or several linked modules) may cause the computing device 130 to provide the described functionality.

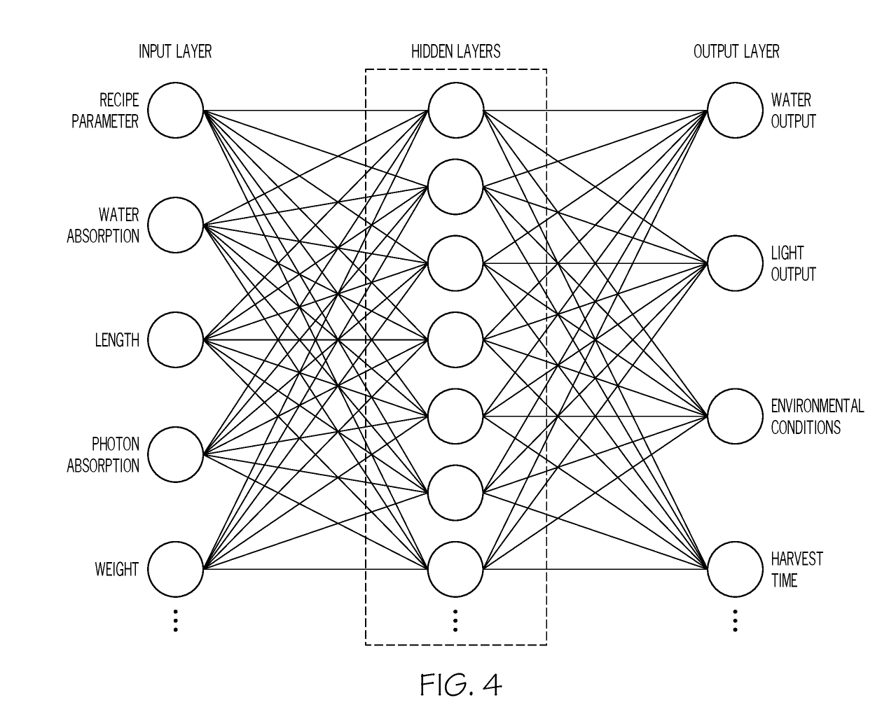

FIG. 4 depicts a neural network node configuration for self-learning in a grow pod 100, according to embodiments described herein. As illustrated, the learning logic 244b may be configured as a neural network or other learning machine. The learning logic 244b may thus have an input layer, one or more hidden layers, and an output layer. The input layer may receive inputs from one or more sensors or other sources, such as data related to a recipe, data related to water absorption by a plant, data related to length of a plant, data related to photon absorption by a plant, data related to weight of a plant, etc. The input layer thus may receive inputs that may be used in learning adaptations to a recipe to more effectively grow the desired plant.

The hidden layers may include a plurality of interconnected nodes that strengthen or weaken connections based on successful or unsuccessful results. There may be one or more layers, depending on the complexity and overall functionality of the system. The output layer may include nodes associated with the changes that may be made to the system to alter the recipe. These nodes may include water output, light output, environmental conditions, harvest time, etc. The output layer nodes may thus be applied to the recipe (such as via the recipe logic 244a to alter a recipe.

It should be understood that while many neural networks may utilize a training phase to improve a task, embodiments described herein utilize this training phase to improve plant growth. As such, once the neural network is trained, embodiments may be configured to cease learning, to prevent overtraining. Similarly, other embodiments may be configured as a three dimensional neural network or other configuration that is resistant to overtraining.

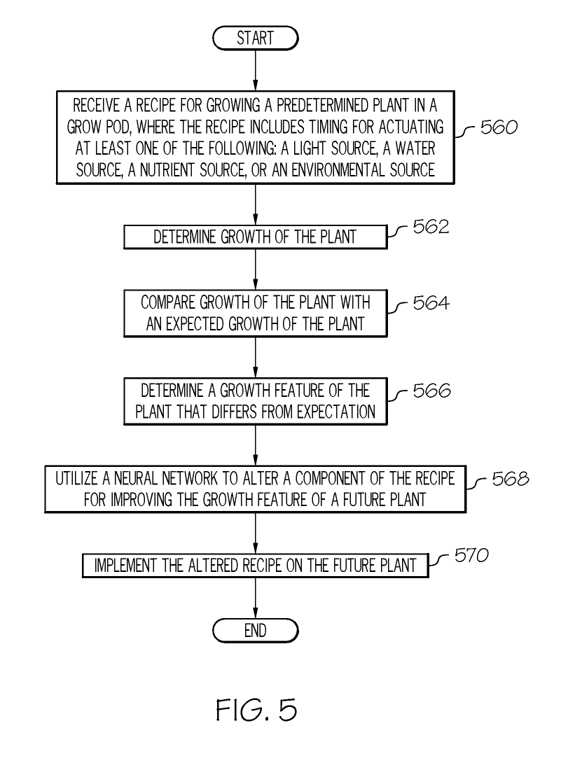

FIG. 5 depicts a flowchart for self-learning in a grow pod 100, according to embodiments described herein. As illustrated in block 560, a recipe for growing a predetermined plant in a grow pod 100 may be received, where the recipe includes timing for actuating at least one of the following: a light source, a water source, a nutrient source, or an environmental source. In block 562, growth of a plant may be determined. In block 564, the growth of the plant may be compared with an expected growth of the plant. In block 566, a growth feature of the plant that differs from the expectation may be determined. A growth feature may include fruit output, plant height, plant girth, weight, and/or other subset of overall plant growth. In block 568, a neural network may be utilized to alter a component of the grow recipe for improving the growth feature of a future plant. In block 570, the altered recipe may be implemented on the future plant.

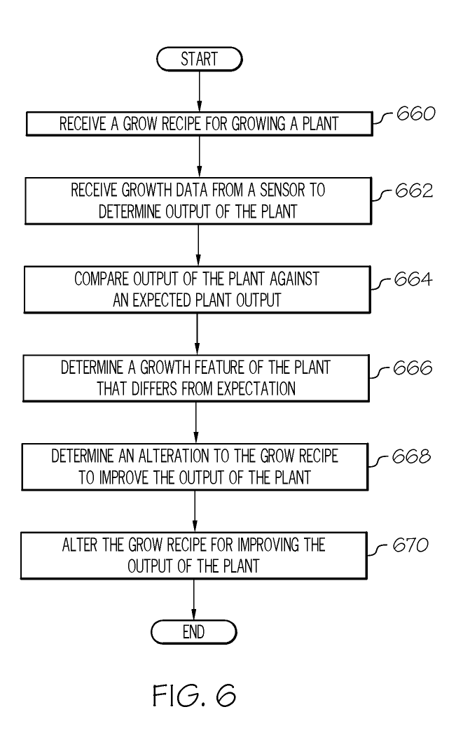

FIG. 6 depicts a flowchart for self-learning and adjusting a grow recipe, according to embodiments described herein. As illustrated in block 660, a grow recipe may be received for growing a plant. In block 662, growth data from a sensor may be received for determining output of the plant. Determining growth data may include determining a growth feature of the plant, such as height, height change, width, width change, color, color change, leaf output, fruit output, etc. Additionally, an expected plant output may be determined. The expected plant output may be received from the computing device 130 and/or determined based on past results.

In block 664, output of the plant may be compared against the expected plant output. In block 666, a determination may be made regarding a growth feature of the plant that differs from the expectation. In block 668, an alteration of the grow recipe may be determined to improve the output of the plant. As an example, the alteration may be a random alteration or random variation. In some embodiments, the alteration may be determined first based on an analysis on the deficient growth feature. If leaf output is deficient (and desired), embodiments may alter the grow recipe such that the environmental affecters that improve leaf growth are changed. Again, depending on the embodiment, this may be determined from past results and/or received from the computing device 130. In block 670, the grow recipe may be altered for improving the output of the plant. In some embodiments, the computing device 130 may communicate the alteration to a remote computing device 254 for implementation by the remote grow pod 200 from FIG. 2.

After the alteration to the grow recipe is received, some embodiments may receive additional growth data from the sensor to determine whether the alteration to the grow recipe resulted in an improved output of the plant. These embodiments may additionally compare the additional growth data with the growth data to determine whether the alteration to the grow recipe improved plant output and, in response to determining that the alteration to the grow recipe did not improve the output of the plant, again alter the grow recipe. If the alteration did improve the plant output, the alteration may be stored for future use and/or sent to the remote grow pod 200 and/or the remote computing device 254 from FIG. 2.

Additionally, some embodiments may receive wear data associated with a component of the grow pod 100. The component may include at least one of the following: the cart 104, the track 102, the environmental affecter, the sensor, and/or other component. Additionally, embodiments may determine a different alteration to the grow recipe to improve longevity of the component and/or the grow pod 100 as a whole.

As illustrated above, various embodiments for self-learning in a grow pod are disclosed. These embodiments may allow a user to upload or otherwise input a grow recipe into a grow pod, where the recipe has one or more commands for light, water, nutrient, environmental, etc. to grow a plant according to a predetermined standard. Embodiments may utilize the recipe; measure the plant growth according to an expectation; and modify the recipe, based on deviation of the actual plant growth from the expectation.

Accordingly, embodiments may include a system and/or method for self-learning in a grow pod that include a growth sensor that senses growth of a feature of a plant in the grow pod; an output sensor that senses outputs of the grow pod for growing the plant; and a computing device that receives a recipe for growing the plant; receives data from the growth sensor; receives data from the output sensor; determines an alteration to the recipe for improving an aspect of plant growth; and implements the change to the recipe.

While particular embodiments and aspects of the present disclosure have been illustrated and described herein, various other changes and modifications can be made without departing from the spirit and scope of the disclosure. Moreover, although various aspects have been described herein, such aspects need not be utilized in combination. Accordingly, it is therefore intended that the appended claims cover all such changes and modifications that are within the scope of the embodiments shown and described herein.

It should now be understood that embodiments disclosed herein include systems, methods, and non-transitory computer-readable mediums for self-learning in a grow pod. It should also be understood that these embodiments are merely exemplary and are not intended to limit the scope of this disclosure.

What is claimed is:

1. An assembly line grow pod for self-learning comprising:
a cart that houses a plant for growth;
a track that receives the cart, wherein the track causes the cart to traverse the assembly line grow pod along a predetermined path;
an environmental affecter for providing sustenance to the plant;
a sensor for monitoring an output of the plant; and
a computing device that stores logic that causes the assembly line grow pod to perform at least the following:
receive growth data from the sensor to determine the output of the plant;
compare the output of the plant against an expected plant output;
determine an alteration to a grow recipe to improve the output of the plant; and
alter the grow recipe for improving the output of the plant.

2. The assembly line grow pod of claim 1, wherein the environmental affecter includes at least one of the following: a light source, a watering device, a nutrient dispensing device, a temperature control device, a humidity control device, a pressure control device, or an airflow control device.

3. The assembly line grow pod of claim 1, wherein the logic further causes the computing device to communicate the alteration to the grow recipe to a remote computing device for implementation by a remote grow pod.

4. The assembly line grow pod of claim 1, wherein the logic further causes the assembly line grow pod to perform at least the following:
receive additional growth data from the sensor to determine whether the alteration to the grow recipe resulted in an improved output of the plant;
compare the additional growth data with the growth data to determine whether the alteration to the grow recipe improved the output of the plant; and
in response to determining that the alteration to the grow recipe did not improve the output of the plant, again alter the grow recipe.

5. The assembly line grow pod of claim 1, wherein the logic further causes the computing device to perform at least the following:
receive wear data associated with a component of the assembly line grow pod, wherein the component includes at least one of the following: the cart, the track, the environmental affecter, or the sensor; and
determine a different alteration to the grow recipe to improve longevity of the component.

6. The assembly line grow pod of claim 1, wherein determining the alteration to the grow recipe includes determining a random variation to the grow recipe.

7. The assembly line grow pod of claim 1, wherein the output of the plant includes at least one of the following: plant growth, root growth, leaf growth, stalk growth, fruit growth, flower growth, protein production, chlorophyll production, or seed success rate.

8. A system for self-learning in a grow pod comprising:
a tray that receives a plurality of seeds and for growing the plurality of seeds into respective plants;
an environmental affecter for providing sustenance to the plurality of seeds;
a sensor for monitoring a plant output; and
a computing device that stores logic that causes the system to perform at least the following:
receive growth data from the sensor to determine the plant output;
compare the plant output against expected plant output;
determine an alteration to a grow recipe to improve the plant output; and
alter the grow recipe for improving the plant output and for improving a plant output of future plants.

9. The system of claim 8, wherein the environmental affecter includes at least one of the following: a light source, a watering device, a nutrient dispensing device, a temperature control device, a humidity control device, a pressure control device, or an airflow control device.

10. The system of claim 8, wherein the grow recipe causes the computing device to control the environmental affecter and movement of the tray along a track.

11. The system of claim 8, further comprising a remote computing device, wherein the logic further causes the computing device to communicate the alteration to the grow recipe to the remote computing device for implementation by a remote grow pod.

12. The system of claim 8, wherein the logic further causes the system to perform at least the following:
receive additional growth data from the sensor to determine whether the alteration to the grow recipe resulted in an improved plant output of the future plants;
compare the additional growth data with the growth data to determine whether the alteration to the grow recipe improved the plant output of the future plants; and
in response to determining that the alteration to the grow recipe did not improve the plant output, again alter the grow recipe.

13. The system of claim 8, wherein the logic further causes the computing device to perform at least the following:
receive wear data associated with a component of the grow pod; and
determine a different alteration to the grow recipe to improve longevity of the component of the grow pod.

14. The system of claim 8, wherein altering the grow recipe includes making a random alteration to the grow recipe.

15. The system of claim 8, wherein the plant output includes at least one of the following: plant growth, root growth, leaf growth, stalk growth, fruit growth, flower growth, protein production, chlorophyll production, or seed success rate.

16. A system for self-learning comprising:
an assembly line grow pod that includes:
a cart that houses a plant for growth;
a track that receives the cart, wherein the track causes the cart to traverse the assembly line grow pod along a predetermined path;
an environmental affecter for providing sustenance to the plant; and
a sensor for monitoring an output of the plant; and
a computing device that stores logic that causes the system to perform at least the following:
receive growth data from the sensor to determine the output of the plant;
compare the output of the plant against expected plant output;
determine an alteration to a grow recipe to improve the output of a future plant; and
alter the grow recipe for improving the output of the output of the future plant.

17. The system of claim 16, wherein the environmental affecter includes at least one of the following: a light source, a watering device, a nutrient dispensing device, a temperature control device, a humidity control device, a pressure control device, or an airflow control device.

18. The system of claim 16, wherein the logic further causes the system to perform at least the following:
receive additional growth data from the sensor to determine whether the alteration to the grow recipe resulted in improved plant output of the future plant;
compare the additional growth data with the growth data to determine whether the alteration to the grow recipe improved the output of the future plant; and
in response to determining that the alteration to the grow recipe did not improve the output of the future plant, again alter the grow recipe.

19. The system of claim 16, wherein the logic further causes the computing device to perform at least the following:
receive wear data associated with a component of the system, wherein the component includes at least one of the following: the cart, the track, the environmental affecter, or the sensor; and
determine a different alteration to the grow recipe to improve longevity of the component.

20. The system of claim 16, wherein plant output includes at least one of the following: plant growth, root growth, leaf growth, stalk growth, fruit growth, flower growth, protein production, chlorophyll production, or seed success rate.
